(19) United States
(12) Patent Application Publication
(10) Pub. No.: US 2010/0068146 A1
(43) Pub. Date: Mar. 18, 2010

(54) Method for Improving Brain Fitness with Consumable Products

(75) Inventors: Stephanie Luther-Forsstrom, Monroe, NY (US); Paul Thomas Quinlan, Milton Ernest (GB)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 12/212,111

(22) Filed: Sep. 17, 2008

(51) Int. Cl. A61K 49/00; G06Q 30/00; G06Q 90/00; A63F 13/00

(52) U.S. Cl. 424/9.2; 705/2; 463/1; 705/14.12

(57) ABSTRACT

The present invention is directed to a method for improving brain fitness. The method includes the step of providing non-protein amino acid to a consumer prior to playing a brain game.

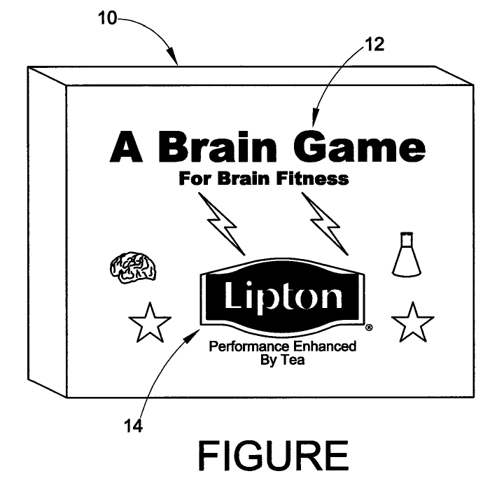

### FIELD OF THE INVENTION

The present invention is directed to a method for improving brain fitness. More particularly, the invention is directed to a method for improving brain fitness by increasing the level of non-protein amino acid in a consumer while the consumer, preferably, is in a relaxed, but ready state. The method of the present invention is expected to enhance, for example, decision making while playing brain games so that the consumer can in the short term become more mentally astute. Moreover, the method of the present invention is expected to prevent cognitive decline and memory loss over a long term, and enhance electrical signals naturally produced by the brain.

### BACKGROUND OF THE INVENTION

Many computer games and other brain exercises are known and commercially available. For example, companies like Sony, Nintendo and Microsoft have made it possible to play games on computers, televisions, and/or hand held electronic devices. Such games include Big Brain Academy for the Nintendo Wii, Brain Challenge for the Microsoft Xbox, as well as Hot Brain for the Sony PlayStation. Other brain games include card games like bridge, gin rummy, and Sudoku as well as board games like chess. Such games may be played, for example, on a computer or via non-electronic means.

Presently, many still believe that games, including electronic games, are not good sources for brain stimulus. In fact, students are often told that computer game playing (like television viewing) can be detrimental to their grades and even their IQ.

Notwithstanding stereotypes often associated with game playing, research on brain games reveals that brain fitness or training (i.e., use of brain games) can enhance the brain's ability to rewire itself and generate new neurons. It is believed, therefore, that such games may result in both short and long term brain benefits, and perhaps even a decrease in Alzheimer symptoms.

Since research is suggesting that brain fitness will provide many benefits, it is desirable to assist a consumer at obtaining good scores or results during a brain game in order to encourage consumers not to quit and to make such game playing part of their daily regiment. This invention, therefore, is directed to a method for improving brain fitness by increasing the level of non-protein amino acid in a consumer while the consumer, preferably, is in a relaxed, but ready state. The method of the present invention is expected to enhance decision making (often by increasing focus and attention) while playing brain games and prevent cognitive decline and memory loss. Such a method is also expected to enhance electrical signals naturally produced by the brain.

### Additional Information

Efforts have been disclosed for making brain games. In U.S. Pat. No. 7,083,420, an interactive handheld apparatus with a display screen is described.

Other efforts have been disclosed for making brain games. In U.S. Pat. No. 5,911,581, an interactive automatic system and technique for measuring and training of mental ability is described.

Even other efforts have been described for making brain games. In U.S. Published Patent Application No. 2008/0138783 A1, a system that can enhance cognitive ability by viewing sequences of images is described.

Other efforts describe means for preparing beverages. In U.S. Patent Application No. 2007/0248737 A1, a process for preparing a tea product with an enhanced level of theanine is described.

None of the additional information above describes a method for improving brain fitness by increasing the level of non-protein amino acid in a consumer while the consumer is, preferably, in a relaxed, but ready state. Moreover, none of the additional information above describes a method that enhances electrical signals naturally produced by the brain, such signals suitable for detection with, for example, an apparatus comprising electrodes.

### SUMMARY OF THE INVENTION

In a first aspect, the present invention is directed to a method for improving brain fitness, the method comprising the steps of:
a) providing non-protein amino acid to a consumer; and
b) having the consumer perform a task in a brain game, the task requiring at least one decision to be made by the consumer.

In a second aspect, the present invention is directed to a method for enhancing electrical signals naturally produced by the brain, the method comprising the steps of:
a) providing non-protein amino acid to a consumer; and
b) measuring brain signals produced by the consumer and subsequent to consuming the non-protein amino acid.

In a third aspect, the present invention is directed to a method for marketing a consumer product comprising non-protein amino acid, the method comprising the steps of:
a) marking a package identifying a brain game with a description of a consumer product comprising non-protein amino acid; and
b) advertising on the package that performance of the brain game will be enhanced by consuming the consumer product comprising non-protein amino acid.

All other aspects of the present invention will more readily become available upon considering the detailed description and example which follow.

Brain fitness, as used herein, means the science or art of maintaining, training and/or gaining cognitive abilities and often through the use of brain games. Brain game, as used herein, means a tool such as but not limited to a sport game (e.g., football, racing), card game, computer game or board game that requires both thinking and decision making. Electrical signals naturally produced by the brain mean waves such as alpha, beta, delta and theta waves (i.e., vibrational pulses) measured in cycles per second or Hertz, all of which are measurable with, for example, an electroencephalograph (EEG) or a magnetoencephalograph (MEG). Such signals are preferably meant to consist essentially of alpha waves, the waves appearing when a consumer is in a wakefulness state where there is a relaxed and effortless alertness. Relaxed, but ready state means the state where alpha waves are strong, a state where there is an effortless alertness as if your eyes are closed and deep thinking or concentration on vivid imagery is very limited. Non-protein amino acid, as used herein, is meant to mean an amino acid that is free or in combined form, but not in a protein. Consumer product as used herein is meant to mean a food product ready to be consumed, like, for example, a beverage (such as coffee, hot chocolate, milk-based product, fruit juice, water, energy drink or tea beverage), spread, sauce, dip, dressing, ice-cream, pasta-based composition, wheat-based composition, meal supplement or replacement drink, or meal supplement or replacement bar, mini-drink, chewing gum, confectionary, or supplement tablet. The providing of non-protein amino acid is preferably achieved by consuming the consumer product.

Tea beverage as used herein is meant to include naturally flavored tea, artificially flavored tea, herbal tea, and especially tea beverages (including decaffeinated tea beverages) with tea solids isolated and recovered from plants like Camellia sinesis, Camilla Asammica, Yabukita or the like. Such a tea beverage may optionally be carbonated and may be sweetened with natural and/or artificial sweeteners. Comprising, as used herein is meant to include consisting essentially of and consisting of.

### BRIEF DESCRIPTION OF THE FIGURES

The subject matter which is regarded as the invention is particularly pointed out and distinctly claimed in the concluding portion of the specification. The invention, however, may be further understood by reference to the following description taken in conjunction with the accompanying drawing FIGURE in which: the FIGURE is a drawing of an illustrative package that identifies a brain game and a consumer product having a non-protein amino acid.

### DETAILED DESCRIPTION OF THE PREFERRED EMBODIMENTS

The only limitations with respect to the type of non-protein amino acids that may be supplied, for example, in the consumer products of this invention are that the non-protein amino acids can provide a benefit to consumers and can be present in consumer product. Often, the non-protein amino acid employed in this invention is plant derived (i.e., isolated therefrom). Illustrative, yet not limiting examples of the types of non-protein amino acids that may be used in this invention include gamma-ethylamino-L-glutamic acid (L-theanine), gamma amino butyric acid (GABA), mixtures thereof or the like. While it is often preferred that the non-protein amino acids used in this invention are plant derived, it is within the scope of this invention to produce (chemically and/or biologically) such non-protein amino acids. Therefore, use of combinations of non-protein amino acids, like theanine, isolated from tea plants as well as theanine that is synthesized is within the scope of this invention.

L-theanine is conventionally isolated from tea plants by extracting the same from dry tea leaves. Production of theanine is also known. For example, Pseudonomas citronellosis GEA, isolated from natural soil, belongs to the genus Pseudomonas, and the species citronellosis and is a theanine producing bacteria with activities that transfer γ-glutamyl groups. With the same, theanine may be biologically produced by using glutaminase derived from the bacteria in a mixture of glutamine and ethylamine at an alkaline pH. Other methods for the synthesis of theanine include those that dehydrate glutamic acid to obtain L-pyrrolidone carboxylic acid which may be added to ethylamine to produce crystals of theanine. Such crystals may be purified via re-crystallization with an alcohol solution. The former method of theanine production is described in European Application No. EP 1544306 A1 and the latter method is described in “The Journal of Tea Science”, 2003 (Vol. 23) (No. 2) 99-104. Another method for making theanine includes the use of glutamic acid derivatives and condensation agents to eventually yield a product which may be stripped of adamantyloxycarbonyl groups and oxy tertiary butyl groups to produce theanine. Such a method is disclosed in more detail in Japanese Application No. 2006083155A. In an especially preferred embodiment, the non-protein amino acid used in the consumer products of this invention is theanine, and particularly, theanine isolated from tea leaves.

Preferably, the consumer product provided to the consumer according to this invention comprises from about 125 ppm to about 20,000 ppm non-protein amino acid (i.e., an amount higher than that found in a conventional tea beverage). In a more preferred embodiment, the food product of the invention comprises at least about 175 ppm non-protein amino acid, and most preferably, from about 200 ppm to about 3,750 ppm non-protein amino acid, including all ranges subsumed therein. While it is within the scope of this invention for all of the non-protein amino acid to be intrinsic, typically extrinsic non-protein amino acid is required in order to achieve the desired concentration within the consumer product of this invention. Often, at least about 25% of the non-protein amino acid employed is extrinsic, and preferably, at least about 65%, and most preferably, at least about 95% of the non-protein amino acid employed in the consumer product of this invention is extrinsic (i.e., added).

In an especially preferred embodiment, the consumer products of the present invention comprise from about 35 to about 4000 ppm, and preferably, from about 150 to about 2500 ppm alkaloid, like caffeine and/or theobromine. Additionally, antioxidants may be added to the consumer products used in the methods of this invention. The only limitations with respect to the same are that the antioxidants are suitable for use in consumer products. Typically, the antioxidants used in this invention are plant (including botanicals) and/or fruit derived, and they include Vitamin C, polyphenols or mixtures thereof. Another antioxidant suitable for use in this invention includes Vitamin E, especially water soluble Vitamin E.

The polyphenols suitable for use in the consumer products used to deliver non-protein amino acids in the methods of this invention include any of those that may be used in a food product and suitable to be formulated with a non-protein amino acid. Examples of the types of polyphenols suitable for use in this invention include those generally classified as flavonoids. Illustrative and non-limiting examples of the types of flavonoids suitable for use in this invention include hesperitin, hesperidin, naringenin, epicatechin, epicatechin gallate, epigallocatechin, epigallocatechin gallate, catechin, gallocatechin, theaflavin, thearubigin, mixtures thereof or the like. Other antioxidants which may be used in this invention include catechol, bearberry, pomegranate and/or rooibos extract. Preferred flavonoids suitable for use in this invention are those which are isolated from citrus fruits (especially from orange juice concentrate), green tea or black tea. The most preferred flavonoids suitable for use in this invention are the flavonoids isolated from green tea, black tea or a mixture thereof.

Often, the consumer product used in the methods of the present invention comprises at least about 25 ppm antioxidants. Preferably, the consumer product of the present invention comprises antioxidant in an amount from about 0.2 to about 1,500 times the amount of non-protein amino acid. Most preferably, the consumer product used in the present invention comprises from about 200 to about 1100 ppm antioxidant, including all ranges subsumed therein. Often, from about 2% to about 100% of the antioxidant used in the present invention is intrinsic. In the case of consumer products with tea solids, typically at least about 85% by weight of the antioxidant used is intrinsic.

When making the consumer product used to provide non-protein amino acid in the methods of the present invention, antioxidant and non-protein amino acid may be added (typically in liquid, powder or concentrate form) as ingredients (in no particular order) and mixed with additional ingredients to produce the desired consumer product. The resulting consumer product (when a beverage) should have a pH of greater than about 3, and preferably, greater than about 3.5, and most preferably, from about 4 to about 6.5, including all ranges subsumed herein. In a preferred embodiment, the consumer product is an energy drink, mini-drink or tea beverage. In a most preferred embodiment, the consumer product is a tea beverage (e.g., black, green, oolong, white or a mixture thereof) with from about 0.5 to about 3.5% by weight tea solids, based on total weight of the beverage.

It is within the scope of this invention to include optional ingredients in the consumer products. Such optional ingredients include chelator (e.g., EDTA), sequestering agent (e.g., sodium hexametaphosphate), colorant, filler (like a dextrin-based compound), preservative, flavor, vitamin (including riboflavin), niacinamide, Vitamin B12, sweetener, fruit juices, surfactant (like sorbitan monolaurate and sorbitan monopalmitate), antifoam or the like.

Still other optional additives which may be used in this invention include herb and nutrient blends like those that comprise at least one of lonicera, forsythia, schizonepeta, ginger, Chinese vitex, isatis root, echinacea or any combination thereof. Amino acids like glutamine and lysine may also be used, if desired, as well as therapeutic agents like those classified as polyfuranosyl-pyranosyl-saccharides recovered from North American Ginseng Fractions as described in U.S. Pat. No. 6,432,454.

Even other optional additives that may be used include high fructose corn syrup, sterol esters, yogurt powder, protein (like milk and whey), dietary fiber (like gum Arabic), ginseng root, guarana, quality stabilizers, acidulants (e.g., citric acid, tartaric acid, and/or phosphoric acid), mixtures thereof or the like. When employed, such optional additives, collectively, make up less than about 55% by weight of the consumer product. In a preferred embodiment, the consumer product used in the methods of the present invention has at least about 70% by weight water, and most preferably, at least about 90% by weight water, based on total weight of the consumer product.

In another especially preferred embodiment, the weight ratio of non-protein amino acid to alkaloid in the food product employed in this invention is from about 4:1 to about 2:1, and preferably, from about 3:1 to about 1:1, including all ratios subsumed therein.

In yet another especially preferred embodiment, the consumer product used in this invention, delivers from about 30 to about 600 mg non-protein amino acid and from about 20 to about 500 mg alkaloid, based on total weight of the consumer product and including all ranges subsumed therein whereby the consumer product has a mass of less than about 510 grams (i.e., a serving size) and further whereby the consumer product is preferably a tea beverage.

The present inventors believe that when administering non-protein amino acid with the consumer product described herein to a consumer (preferably, in a relaxed, but ready state), the consumer will perform better in a brain game, and especially, if the consumer product is consumed within a two (2) hour period (preferably, within a 5 to 60 minute period) and playing of the brain game starts within three (3) hours of consuming the consumer product, and preferably, within about 5 to about 120 minutes, and most preferably, within about 5 to about 60 minutes of consuming the consumer product. In an especially preferred embodiment the brain game is completed within about seven (7) hours of consuming the consumer product, and preferably, within about three (3) hours of consuming the consumer product, where consuming of consumer product with non-protein amino acid preferably means consuming a serving size.

Not to be bound by theory, it is believed that the consumption of non-protein amino acid in the manner described herein will increase brain alpha waves, leaving the consumer better suited to start playing and performing in a brain game that requires thought processes associated with additional brain waves. The surge or increase in alpha waves during, for example, the relaxed, but ready state (and as a result of consuming non-protein amino acid) will not only enhance decision making during the brain game (and get consumers more addicted and excited about playing the game), but also make the detection of the alpha waves (and the change in alpha waves that can occur when the consumer is performing the brain game) clearer and easier by, for example, known techniques such as EEG and/or MEG. Such detection may also be made easier when using publicized devices such as the OCZ Neural Impulse Actuator and the NeuroSky electrode headset, both of which are generally classified as EEG technologies for playing games. These types of devices will benefit from the method of this invention since an increase in alpha waves and better detection thereof should make the monitoring of consumer mental attributes like, for example, facial expressions, emotions, and calmness of mind easier. The better detection of consumer mental attributes will, for example, make brain games that rely on such mental attributes more fun, accurate and addictive, thereby leading to a consumer having improved brain fitness.

In a preferred embodiment, the preferred publicized devices suitable for use with the present invention are those made by Emotiv Systems, an Australian electronics company that develops brain-computer interfaces based on EEG. Emotiv's Emotiv EPOC, is a peripheral for gaming, for example, on Window's PCs. Other and future applications for such technologies include interactive television, accessibility design, market research as well as medicine, security and honesty detection.

Headsets made by Emotiv make it possible for games to be controlled and influenced, for example, a consumer's mind, facial expressions or both. Such headsets use sensors to tune into electrical signals detecting thoughts, feelings and expressions of a consumer. Headsets are typically wireless, and the system contains software to emulate keystrokes. Programs, games and the like are controlled by the headsets and its electrodes that pick up signals naturally produced by the brain. It is the understanding of the present inventors that such signals (especially alpha signals) will be enhanced by the method of the present invention thereby resulting in, for example, more use of brain games and improved brain fitness for consumers. A more detailed description of the types of headsets suitable for use and suitable to benefit from the present invention is described in U.S. Patent Application Nos. 2007/0225585 A1 and 2007/0060831 A1, the disclosures of which are incorporated herein by reference.

Moreover, it is understood that repeated use of non-protein amino acid while performing such brain games will, surprisingly, improve long term cognitive abilities and the ability of the consumer to learn how to control brain/computer interfaces.

It is also the understanding of the present inventors that consumption of non-protein amino acid prior to the brain game will improve sustained attention during the game. Such a result is important since brain games can last for multiple minutes and/or hours, requiring the consumer to maintain attention throughout the game. The games can also require attention switching between auditory and visual domains and it is understood that non-protein amino acid will aid the consumer with attention switching.

Turning to the figure, shown is a package 10 having a brain game name 12 thereon. The package 10 further comprises the name (e.g., advertisement) of an illustrative consumer product 14 (having non-protein amino acids) that is encouraged for use when a decision is made to play the game identified on the package 10. Thus, it is within the scope of the present invention to mark a consumer product package with a game name or a game package with a consumer product name. In a most preferred embodiment, the consumer product described herein should be prepared and supplied by Unilever under the Lipton® Tea Brand.

The packaging for the consumer product described herein typically is a can, bottle, pouch or drinking box.

The example which follows is provided to facilitate an understanding of the present invention. The invention is not intended to limit the scope of the inventions claimed.

### EXAMPLE

Prior to playing brain games, such as chess, and during a relaxed, but ready state, consumers may be provided with a serving of the beverage containing 100 mg theanine and 45 mg of caffeine. The consumers may be instructed to consume the tea beverage in about a fifteen (15) minute period and to start playing the brain games within thirty (30) to sixty (60) minutes of consuming the tea beverage.

It is expected that the consumption of the tea beverage with the non-protein amino acid (e.g., theanine) will enhance the performance of the consumers playing such games.

We claim:

1. A method for improving brain fitness, the method comprising the steps of:
(a) providing non-protein amino acid to a consumer; and
(b) having the consumer perform a task in a brain game, the task requiring at least one decision to be made by the consumer.

2. The method according to claim 1 wherein the non-protein amino acid is theanine, GABA or a mixture thereof.

3. The method according to claim 1 wherein the non-protein amino acid is provided to the consumer in a consumer product, and the brain game requires sustained attention, or visual and auditory attention, or both.

4. The method according to claim 3 wherein the consumer product is a beverage, spread, sauce, dip, dressing, ice-cream, pasta-based composition, wheat-based composition, meal supplement or replacement drink, meal supplement or replacement bar, mini-drink, chewing gum, confectionary or supplement tablet.

5. The method according to claim 1 wherein the non-protein amino acid is provided during a relaxed, but ready state.

6. The method according to claim 1 wherein the consumer product further comprises an alkaloid.

7. The method according to claim 6 wherein the alkaloid is caffeine, theobromine or a mixture thereof.

8. The method according to claim 7 wherein the consumer product comprises from about 35 to about 4000 ppm alkaloid and from about 125 ppm to about 20,000 ppm non-protein amino acid.

9. The method according to claim 1 wherein the non-protein amino acid is provided in a serving of tea beverage comprising from about 30 to about 600 mg non-protein amino acid and from about 20 to about 500 mg alkaloid in less than about 510 grams of the tea beverage.

10. The method according to claim 9 wherein the tea beverage further comprises flavonoids isolated from green tea, black tea or both.

11. The method according to claim 1 wherein the non-protein amino acid is provided in a consumer product and to the consumer within a two hour period and playing of the brain game starts within three hours of consuming the consumer product.

12. A method for enhancing electrical signals naturally produced by the brain, the method comprising the steps of:
(a) providing non-protein amino acid to a consumer; and
(b) measuring brain signals produced by the consumer and subsequent to consuming the non-protein amino acid.

13. The method according to claim 12 wherein the brain signals comprise alpha, beta, delta and theta waves.

14. The method according to claim 12 wherein the brain signals consist essentially of alpha waves and changes thereof.

15. The method according to claim 12 wherein non-protein amino acid is provided in a serving of tea beverage comprising from about 30 to about 600 mg non-protein amino acid and from about 20 to about 500 mg alkaloid in less than about 510 grams of the tea beverage.

16. The method according to claim 12 wherein the brain signals are detected with EEG, MEG or both.

17. A method for marketing a consumer product comprising non-protein amino acid, the method comprising the steps of:
a) marking a package identifying a brain game with a description of a consumer product comprising non-protein amino acid; and
b) advertising on the package that performance of the brain game will be enhanced by consuming the consumer product comprising non-protein amino acid.

18. The method for marketing a consumer product according to claim 17 wherein the package contains a brain game and is marked with a description of a consumer product.

19. The method for marketing a consumer product according to claim 17 wherein the package contains a consumer product, describes the consumer product and identifies a brain game.

20. The method for marketing a consumer product according to claim 17 wherein the consumer product is a tea beverage.
